(19) United States
(12) Patent Application Publication
(10) Pub. No.: US 2011/0118554 A1
(43) Pub. Date: May 19, 2011

(54) DEVICE-ASSISTED SOCIAL NETWORKING FOR HEALTH MANAGEMENT

(75) Inventors: Christopher J. Stakutis, Concord, MA (US); Philip Lawrence, Bolton, MA (US)

(73) Assignee: COMPUTER ASSOCIATES THINK, INC., Islandia, NY (US)

(21) Appl. No.: 12/620,196

(22) Filed: Nov. 17, 2009

(51) Int. Cl. A61B 5/00; G06Q 99/00; G06Q 50/00

(52) U.S. Cl. 600/300; 705/319; 705/2

(57) ABSTRACT

Device-assisted social networking for health management, in one aspect, collects health related information associated with one or more users, performs analysis and publishes data resulting from the analyzing to a social networking website associated with one or more users, wherein the data may be shared among the users in a group for peer encouragement in habits that would improve the users' health conditions.

### FIELD OF THE INVENTION

The present disclosure relates generally to computer system and software, and more particularly to device-assisted social networking for health management.

### BACKGROUND OF THE INVENTION

Factors such as aging, unhealthy dietary habits and lack of proper fitness care can lead to increases in chronic diseases such as diabetes, heart disease, obesity which leads to other diseases, and others. Health monitoring devices are made available on the market that allow patients or the like users to be able to measure health related information at home or on their own regularly without the aid of a health professional. Such devices may include heart or pulse rate measuring device, blood pressure measuring devices, blood sugar level identifiers, or even the old-fashioned weight scale that allows one to measure the body weight. Patients or the like users then may record the measured data, report it and discuss their health conditions with the doctor or another professional at their next visitation or appointment.

Keeping track of one's health information, maintaining healthy habits and activities and regular checkups all help in proactively preventing more serious ailments and illness in human lives. Often times, however, healthy individuals are too busy with other matters and do not have enough motivation to follow the well-being health habits. It is not easy an easy task to convince the people to adjust or change their schedules and habits to fit in more well-being and healthier activities and behaviors. Usually, only when it is discovered that one is inflicted with ailments, do people seek treatments. Accordingly, what is desirable is for a way in which to convince, motivate and/or encourage people to include healthy regimen in their schedule.

Social networking sites (e.g., World Wide Web sites) provide utilities that connect people with other people, for example, a group of friends, people who work together, or those that have other commonalities, for example, remotely online from the user devices or computers. Online social networking sites usually include access privileges for the users so that some privacy may be maintained. Once granted access, the user can begin to socialize. One commonly used social networking site is Facebook™.

### BRIEF SUMMARY OF THE INVENTION

A method and system for device-assisted social networking for health management are provided. The method, in one aspect, may include collecting at a server executing one or more computer processors, health related information associated with a user, analyzing, by a computer processor, the health related information associated with a user, and publishing data resulting from the analyzing to a social networking website associated with the user. The data may be presented in a manner as to provide peer encouragements for adapting healthy habits and/or behavior.

A system for device-assisted social networking for health management, in one aspect, may include a processor and a server module operable to execute on the processor. The server module may be operable to collect health related information associated with a user, and analyze the health related information associated with a user. The server module may be further operable to publish data resulting from the analyzing to a social networking website associated with the user. The analyzed data may be published and shared in a manner as to provide peer encouragements for adapting healthy habits and/or behavior.

A computer readable storage medium storing a program of instructions executable by a machine to perform one or more methods described herein also may be provided.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1 is an architectural diagram illustrating components for system of the present disclosure in one embodiment.

FIG. 2 is a flow diagram illustrating a method in one embodiment for device-assisted social networking health management.

### DETAILED DESCRIPTION

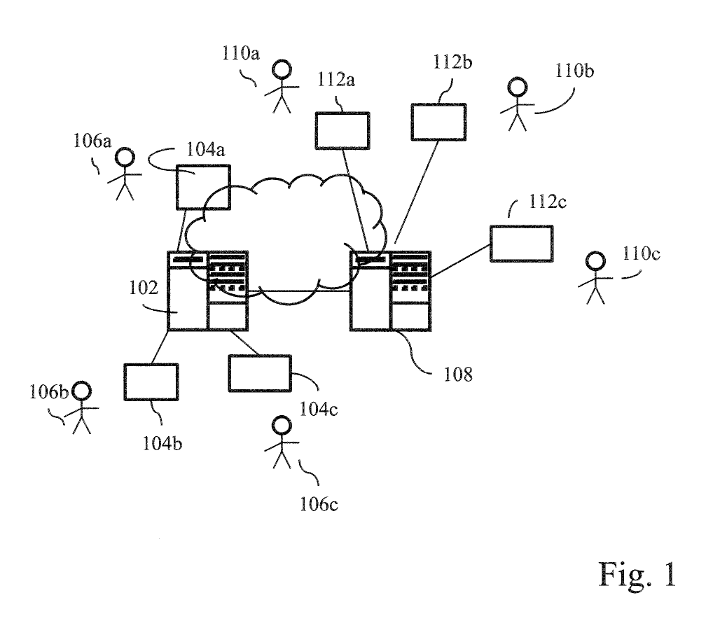

A subscribed social networking tool is enhanced with end-user device monitoring equipment or data entry device and provides a health management solution. FIG. 1 is an architectural diagram illustrating components for system of the present disclosure in one embodiment. A computer system, for example a server 102 connects to a plurality of remote and/or local end user devices (104a, 104b, 104c, etc.) and collects information from the devices (104a, 104b, 104c, etc.). The end user devices communicate data related to medical or health conditions about the respective users at the end user devices to the server 102. The users, of course, need not be tied to the specific devices when submitting data. For example, the users may enter data using their user identifiers or login information recognized by the server 102 from different end user devices.

An end user device (e.g., 104a, 104b, 104c, etc.) may be any device enabled to connect to the server 102 and communicate data to the server 102. Examples of the end user devices (104a, 104b, 104c, etc.) may include, but are not limited to, data entry device that a user may enter their health information, biometric measurement data that measure information about the person's physical conditions, and other devices. For instance, an end user device may include, but is not limited to, a personal computer (PC), a laptop, personal digital assistant (PDA) device, that the user can use to enter the health related information and transmit the data to the server. The end user device may also be a health monitoring equipment or other devices that measure health information for associated users and directly transmit the data to the server 102, for instance, without the user having to enter the data. Weight scales may be available that can transmit their readings, for example, via a wireless connection such as Bluetooth™ to a nearby computer that in turn transmits that data into an Internet based server (e.g., 102). Other devices may include a pulse-oximeter for determining blood O2 saturation, wireless glucometers, wired or wireless blood pressure cuffs, and many more such devices that can transmit data related to physical conditions of a person. Even a mobile device such as a mobile wireless phone may be able to send “motion data” (steps, exercise data) to the server 102. Yet other examples may include various equipments in an exercise facility enabled to transmit data.

The server 102 may be implemented as a computing cloud or reside in a data center, data farm, or a proprietary server associated with a specific health care management system, or other, and may be associated with health care management system or other health monitoring center or others. The end user devices (104a, 104b, 104c, etc.) may periodically or continually send health related measurements of the respective users (106a, 106b, 106c, etc.) to the server 102 associated with respective users at those end user devices (104a, 104b, 104c, etc.). The data may include but is not limited to blood sugar level, blood pressure, pulse rate, heart rate, weight, and any other health related information. Capturing of the data may be transparent as possible allowing for high rate of data capture. For example, convenient wireless devices may make it simple for a user to sample their biometric data and not have to worry about transcribing a result into any software. Such automation makes it that much simpler for people to easily produce data about their health.

The server 102 receives the data and performs analysis on the data for presenting it to the users. For instance, an analysis module running on the processor acting as a server 102 may perform the data analysis. The data on the server may be automatically formatted for a variety of reports that can be easily explored by either the patient or the care giver (for example, charts of weight history, which can be view by on-line web-based portals or specialized display devices or social networking sites). The server may also perform some analysis of the data and determine that the patient should receive a message (a text message, email, or a note on a social networking site) to encourage behavior changes or to reward progress. The information at the server 102 may be managed by doctors or other health professionals. Because the information may be managed by doctors and other health professionals, users or patients may be more willing to participate.

The server 102 may be connected to one or more social networking sites of which the one or more users (106a, 106b, 106c, etc.) are members. The server computer 102 presents the health data it analyzed and presents them to the users (106a, 106b, 106c, etc.) in a manner that would encourage the users to change their behavior in a good healthful way. Presenting the data in a useful and informative manner on a social networking site may spark a friendly competition and/or peer encouragements among the participants of the social networking group, such that the persons in the group may each want to be more proactive in living a healthy life. For instance, sociologists have observed that people are typically unwilling to change their behavior unless encouraged or witness by a set of peers. Social networking connects people together, for instance, for sharing information and often in a “group” setting. It is expected that group “notice” (recognition) may effect how an individual behaves.

For example, a user may log on via the user interface such as the web browser from his or her location to the social networking site 108. The user then may communicate or network with other users (e.g., 110a, 110b, 110c, etc.) about his or her health conditions as analyzed and presented to the user's page, for example on the respective users' devices (e.g., 112a, 112b, 112c, etc.). Users and/or patients can see each other's data and health-progress. As a result, they can earn rewards and/or points, or potentially warnings. The sever 102 may automatically send messages to others in a particular group about the progress (positive or negative) about individual users, to further encourage their positive participation. The users (110a, 110b, 110c, etc.) at the social networking site 108 may be the same set of users (106a, 106b, 106c, etc.) that use the service of the server 102, or an overlapping set of users (i.e., some the same and others not).

It should be noted that the user's health-related data that is shared among the social networking group need not be raw personal or private data. Rather, the server 102 may embody the raw data into the form of relative data, percentage data, normalization, etc., for example, so that the individuals can still maintain their privacy. For instance, the data shown may be in the form of percentage of improvement or decline from the last measurement data. Another example may be overall statistics based on the group data. In this way, a user or patient's private health-related data need not be compromised.

Social networking, for example, informal social networks are inexpensive and popular, and would serve as an effective peer encouragement device. The presence of the group/forum may allow for the high feedback on the system overall and thus may allow for rapid increase in the number of people participating. More people participate may lead to more people adapting a more healthful way of living. This way, many ailments and diseases may be prevented before they inflict the people. A social networking site may be a site implemented specifically for use with the sharing of the health related information as disclose herein. Alternatively or additionally, a social networking site may be a known or existing site such as the Facebook™.

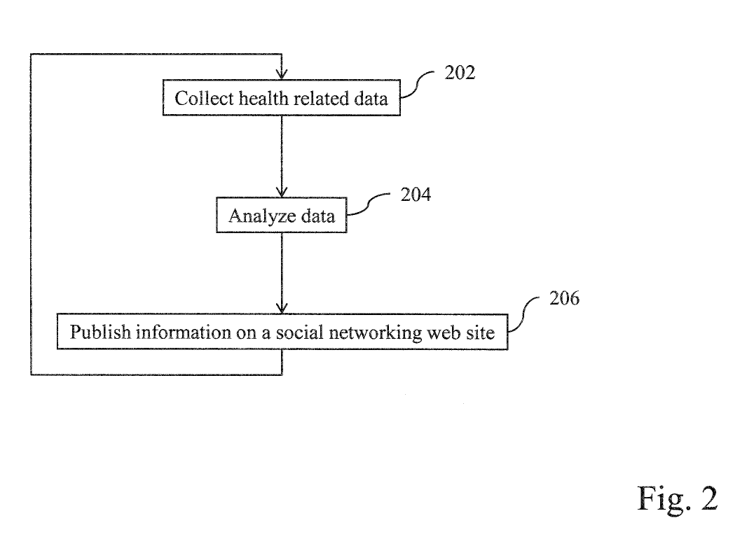

FIG. 2 is a flow diagram illustrating a method in one embodiment for device-assisted social networking health management. At 202, a computer processor acting as a server for device-assisted social networking health management system collects information from a plurality of users, connected remotely or locally. The information may relate to various health conditions of the users, such as body weight, heart rate, pulse rate, blood pressure, blood sugar level, skin color, degree of fatigue the user is experiencing that the time, and others. The information may be collected periodically at a predetermined interval, and/or at any time the user decides to provide the data. For instance, the user may connect to the server and enter their health related information at their convenience. If, for instance, the server detects that the user has not submitted any data in certain period of time (for example, no data came in from certain user in the last week), the server may prompt the user or otherwise notify the user to submit the data. The prompt or notification may be in a form of an email, text message such as SMS text message, a pop-up reminder if the user happens to log on the server associated web site or the like. Other ways of notifying the user is possible.

At 204, the server may analyze the collected data, and for example, determine what health condition related to each user has improved, degraded, or remained the same, over any specified or predetermined time period, between the last time the user specified the data and the current time, and others. The server may determine, for example, that the user has not produced data recently (for example, a body weight reading) and encourage or warn as a result. Similarly, based on various data received and known conditions of the patient, the server may assign a series of questions-and-answers to be asked of the patient to better determine their health (perhaps a diabetic is losing sensitivity in their toes and this is an indication of an infection that could lead to amputation).

At 206, the server may present this data on a social networking website of which the user is a member. The data may be presented in a graph form showing changes over time, in a table format, or in any other form. The data also may be shown with additional hints or directions as to how the user's health condition may improve, for instance, incrementally if the user were to follow those hints and directions. Again, the user's raw or sensitive data need not be shown so as to preserve the user's privacy. Rather, relative data may be used.

The user may decide to share this information with the user's private group or the like on the social networking website. Some users/patients may find it very compelling if people in their care group (parents, kids, relatives) know of their changing health status. Various health data can be automatically “shared” without effort and thus allow a more fluid connection to the care group which in turn may help the individual focus on their health.

The various functionalities and modules of the systems and methods of the present disclosure may be implemented or carried out distributedly on different processing systems or on any single platform, for instance, accessing data stored locally or distributedly on the network.

Various aspects of the present disclosure may be embodied as a program, software, or computer instructions embodied or stored in a computer or machine usable or readable medium, which causes the computer or machine to perform the steps of the method when executed on the computer, processor, and/or machine. A program storage device readable by a machine, tangibly embodying a program of instructions executable by the machine to perform various functionalities and methods described in the present disclosure is also provided.

The system and method of the present disclosure may be implemented and run on a general-purpose computer or special-purpose computer system. The computer system may be any type of known or will be known systems and may typically include a processor, memory device, a storage device, input/output devices, internal buses, and/or a communications interface for communicating with other computer systems in conjunction with communication hardware and software, etc.

The terms “computer system” and “computer network” as may be used in the present application may include a variety of combinations of fixed and/or portable computer hardware, software, peripherals, and storage devices. The computer system may include a plurality of individual components that are networked or otherwise linked to perform collaboratively, or may include one or more stand-alone components. The hardware and software components of the computer system of the present application may include and may be included within fixed and portable devices such as desktop, laptop, and/or server. A module may be a component of a device, software, program, or system that implements some “functionality”, which can be embodied as software, hardware, firmware, electronic circuitry, or etc.

The embodiments described above are illustrative examples and it should not be construed that the present invention is limited to these particular embodiments. Thus, various changes and modifications may be effected by one skilled in the art without departing from the spirit or scope of the invention as defined in the appended claims.

We claim:

1. A method for device-assisted social networking for health management, comprising:
collecting at a server executing one or more computer processors, health related information associated with a user;
analyzing, by a computer processor, the health related information associated with a user; and
publishing data resulting from the analyzing to a social networking website associated with the user.

2. The method of claim 1, wherein the user is located remotely from the computer processor.

3. The method of claim 1, wherein the health related information associated with a user is collected automatically from biometric devices, the biometric devices enabled to measure the health related information associated with the user.

4. The method of claim 3, wherein the biometric devices are located remotely from the computer processor.

5. The method of claim 1, wherein the health related information associated with a user is submitted by the user by the user entering the health related information on a user device and transmitting the health related information to the computer processor.

6. The method of claim 1, wherein the publishing includes presenting the data in a graph format showing changes to health condition of the user over a predetermined period of time.

7. The method of claim 1, wherein the publishing includes providing one or more recommendations for improving health conditions of the user.

8. The method of claim 1, wherein the publishing includes sharing the data with a selected group of users in the social networking website.

9. A system for device-assisted social networking for health management, comprising:
a processor;
a server module operable to execute on the processor, and further operable to collect health related information associated with a user, and analyze the health related information associated with a user, the server module further operable to publish data resulting from the analyzing to a social networking website associated with the user.

10. The system of claim 9, further including:
a social networking web site operable to allow one or more users to interact in a group and share data relating to health of the one or more users.

11. The system of claim 9, wherein the server module is operable to present the analyzed health related information on the social networking website to provide peer encouragement in adjusting user's health behavior.

12. A computer readable storage medium storing a program of instructions executable by a machine to perform a method of device-assisted social networking for health management, comprising:
collecting at a server executing one or more computer processors, health related information associated with a user;
analyzing, by a computer processor, the health related information associated with a user; and
publishing data resulting from the analyzing to a social networking website associated with the user.

13. The computer readable storage medium of claim 12, wherein the user is located remotely from the computer processor.

14. The computer readable storage medium of claim 12, wherein the health related information associated with a user is collected automatically from biometric devices, the biometric devices enabled to measure the health related information associated with the user.

15. The computer readable storage medium of claim 14, wherein the biometric devices are located remotely from the computer processor.

16. The computer readable storage medium of claim 12, wherein the health related information associated with a user is submitted by the user by the user entering the health related information on a user device and transmitting the health related information to the computer processor.

17. The computer readable storage medium of claim 12, wherein the publishing includes presenting the data in a graph format showing changes to health condition of the user over a predetermined period of time.

18. The computer readable storage medium of claim 12, wherein the publishing includes providing one or more recommendations for improving health conditions of the user.

19. The computer readable storage medium of claim 12, wherein the publishing includes sharing the data with a selected group of users in the social networking website.
